(19) United States
(12) Patent Application Publication
(10) Pub. No.: US 2005/0030473 A1
(43) Pub. Date: Feb. 10, 2005

(54) Apparatus and method for determining intraocular pressure and corneal thickness

(75) Inventors: Corinn C. Fahrenkrug, Liverpool, NY (US); Chris R. Roberts, Skaneateles, NY (US); Raymond A. Lia, Auburn, NY (US); Ervin Goldfain, Syracuse, NY (US); Jeffrey Perkins, Tully, NY (US); Allan I. Krauter, Skaneateles, NY (US); Andrew J. Kugler, Albany, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc., Skaneateles Falls, NY (US)

(21) Appl. No.: 10/865,032

(22) Filed: Jun. 10, 2004

Related U.S. Application Data
(60) Provisional application No. 60/478,564, filed on Jun. 12, 2003.

(51) Int. Cl. A61F 2/16

(52) U.S. Cl. 351/200

(57) ABSTRACT

The invention includes methods and apparatus for measuring (Intraocular Pressure) IOP and (Corneal Thickness) CT for diagnosis and for monitoring a condition of health of an eye. The apparatus uses measurements selected from at least one of IOP and a CT, and a plurality of IOP measurements to deduce an interpretive result indicative of a condition of an eye. In another embodiment, the apparatus measures a relationship between an applanated area of variable size and a selected one of an applied force and an applied pressure. The relationship is used to deduce an interpretive result indicative of the condition of an eye. The result can be displayed to a practitioner. The practitioner uses the result to determine a course of treatment as necessary, and to monitor the effect of a course of treatment on the state of health of the eye over time.

### CROSS REFERENCE TO RELATED APPLICATIONS

This application claims priority to and the benefit of co-pending U.S. Provisional Patent Application Ser. No. 60/478,564, filed Jun. 12, 2003, which application is incorporated herein by reference in its entirety.

### FIELD OF THE INVENTION

The invention relates to intraocular pressure and corneal thickness measurements in general and particularly to systems and methods for intraocular pressure assessment that employ a plurality of interrelated techniques.

### BACKGROUND OF THE INVENTION

The classical measurement of intraocular pressure (hereinafter “IOP”) is a mechanical tonometry method invented by Goldmann in the early 1950s. The Goldmann applanation method measures the force required to flatten a certain area of the cornea. A fine strip of paper that is stained with an orange colored dye (fluorescein) is touched to the side of the eye. The dye stains the front part of the eye to help with the examination. The dye rinses out of the eye with tears. An anesthetic drop is also placed in the eye. A slit-lamp is placed in front of a person to be tested. The person rests his or her chin and forehead on supports that hold the person's head steady. The slit lamp is moved forward until the tonometer touches the cornea. The light is usually a blue circle and there is generally no discomfort associated with the test. Using the blue light as a signal, the health care provider looks through the eyepiece on the lamp and adjusts the tension of the tonometer until an area of 7.3542 mm² is applanated. 7.3542 mm² is the area of a piston of 3.06 mm diameter, and is believed to be the smallest area needed for accurate results, but which causes an increase in apparent IOP of 2.5%. The force that is required to applanate the area of the eye is measured. Based on the relation Pressure=Force/Area, an area of 7.3542 mm² is the area for which a 1 gram force is equivalent to an IOP of 10 mm of mercury (Hg), and a 2 gram force corresponds to an IOP of 20 mm Hg. One of the reasons why this area was selected has to do with the convenience of calculation of the operator of the instrument.

Airpuff tonometry is a noncontact method of measuring IOP. In this method, the person's chin rests on a padded stand. The person is asked to stare straight into the examining instrument. The instrument uses a bright light directed to the eye to properly align the instrument and to make measurements. A puff of air applies a force to the surface of the eye. The eye responds to the force applied by the air puff. The deflection of the surface of the eye is measured using changes in the light reflected off the cornea. The instrument calculates IOP from the deflection caused by the air puff.

A number of problems in measuring IOP have been observed. Some of these relate to diurnal variations in IOP, and variations in IOP as a result of treatment of the eye with laser surgery such as Laser In Situ Keratomiluesis (LASIK) and Photorefractive Keratectomy (PRK). Other problems are thought to arise as a consequence of corneal thickness variations. Based on statistical information, the “standard” thickness of a cornea is taken in the literature as 0.522 mm. Nevertheless, it is known that different individuals have corneas that may be thicker or thinner than the “standard” value and that such deviation in thickness can affect the outcome of a measurement of IOP. Typically, measured IOP is raised as the thickness of a cornea exceeds 0.522 mm and is decreased as the thickness falls below 0.522 mm.

There is a need for systems and methods that provide better measurement of IOP, and for systems and methods that take corneal thickness into account, both for the diagnosis and the management of eye conditions such as glaucoma.

### SUMMARY OF THE INVENTION

In one aspect, the invention relates to an apparatus for producing an accurate value of intraocular pressure of an eye. The apparatus comprises an intraocular pressure measuring device, the intraocular pressure measuring device providing a first intraocular pressure measurement of an eye; a device for making a selected one of a corneal thickness measurement and at least one additional intraocular pressure measurement, separate and distinct from the first intraocular pressure measurement; and a data analysis module that interrelates the first intraocular pressure measurement and the selected one of the corneal thickness measurement and the at least one additional intraocular pressure measurement. An accurate value of intraocular pressure is provided.

In one embodiment, the apparatus further comprises a display module for displaying to a user of the apparatus the accurate value of intraocular pressure. In one embodiment, the data analysis module is further configured to generate an interpretive result indicative of a condition of the eye. In one embodiment, the apparatus further comprises a display module for displaying to a user of the apparatus the interpretive result indicative of a condition of the eye. In one embodiment, the data analysis module is further configured to generate an interpretive result useful to evaluate a condition of the eye. In one embodiment, the data analysis module is further configured to generate an interpretive result useful to manage a condition of the eye. In one embodiment, the intraocular pressure measuring device and the device for making at least one additional intraocular pressure measurement are the same device. In one embodiment, the first intraocular pressure measurement and the at least one additional intraocular pressure measurement are intraocular pressure measurements performed on different regions of the eye. In one embodiment, the different regions comprise regions having different locations on a surface of the eye. In one embodiment, the different regions comprise regions having different surface areas on a surface of the eye. In one embodiment, the intraocular pressure measuring device is a device of the Goldmann type. In one embodiment, the intraocular pressure measuring device is adapted to measure intraocular pressure using a selected one of a first area and at least an additional area. In one embodiment, a change in applanated area with change in a selected one of an externally applied force and an externally applied pressure is used for estimating intraocular pressure. In one embodiment, the intraocular pressure measuring device is a non-contact intraocular pressure measurement device. In one embodiment, the device for measuring the corneal thickness is a selected one of an apparatus passing light transversely through a cornea and an apparatus using a plurality of optical beams reflecting from different surfaces of a cornea.

In another aspect, the invention features a method of producing an accurate value of intraocular pressure of an eye. The method comprises the steps of measuring a first intraocular pressure of an eye; measuring a selected one of a corneal thickness of the eye and at least one additional intraocular pressure of the eye, separate and distinct from the first intraocular pressure; interrelating the first intraocular pressure and the selected one of the corneal thickness and the at least one additional intraocular pressure; and providing an accurate value of intraocular pressure.

In one embodiment, the method further comprises the step of displaying to a user of the method the accurate value of intraocular pressure. In one embodiment, the method further comprises the step of generating an interpretive result indicative of a condition of the eye. In one embodiment, the method further comprises the step of displaying to a user of the method the interpretive result indicative of a condition of the eye. In one embodiment, the method further comprises the step of generating an interpretive result useful to evaluate a condition of the eye. In one embodiment, the method further comprises the step of generating an interpretive result useful to manage a condition of the eye. In one embodiment, the first intraocular pressure measurement and the at least one additional intraocular pressure measurement are performed on different regions of the eye. In one embodiment, the different regions comprise regions having different locations on a surface of the eye. In one embodiment, the different regions comprise regions having different surface areas on a surface of the eye. In one embodiment, the intraocular pressure measurement is performed using a device of the Goldmann type. In one embodiment, the intraocular pressure measurement is performed using a selected one of a first area and at least one additional area. In one embodiment, a change in applanated area with a change in a selected one of an externally applied force and an externally applied pressure is used for estimating intraocular pressure. In one embodiment, the intraocular pressure measurement is performed using a non-contact intraocular pressure measurement device. In one embodiment, the measurement of the corneal thickness is performed using a selected one of a method in which light passes transversely through a cornea and a method using a plurality of optical beams reflecting from different surfaces of a cornea.

In another aspect, the invention relates to an apparatus for obtaining a relationship between a selected one of an applied force and an applied pressure, and a corresponding area of applanation of an eye. The apparatus comprises a driver application section for applying to a surface of an eye a selected one of a variable force and a variable pressure to cause applanation of a portion of the surface of the eye; and a measurement section measuring the selected one of a variable force and a variable pressure, and measuring a corresponding variable area of applanation of the surface of the eye; whereby the apparatus obtains at least one point of a relationship between the area of applanation of the eye and the selected one of a variable force and a variable pressure.

In one embodiment, the apparatus further comprises an analysis module for deducing an accurate value of intraocular pressure. In one embodiment, the apparatus further comprises a display module for displaying to a user of the apparatus an accurate value of intraocular pressure. In one embodiment, the apparatus further comprises a memory in communication with the measurement section, the memory configured to record a database comprising at least one entry, the entry representing a relationship between a selected one of an applied pressure and an applied force and a corresponding area of applanation, the entry correlated with a condition of at least one eye. In one embodiment, the apparatus further comprises a processor for comparing the at least one point of a relationship of area of applanation of the eye corresponding to the selected one of an applied pressure and an applied force with at least one entry in the database comprising at least one relationship between a selected one of an applied pressure and an applied force and a corresponding area of applanation, each entry correlated with a condition of at least one eye. In one embodiment, the apparatus further comprises an output device for reporting an interpretive result indicative of a condition of the eye.

In yet another aspect, the invention features a method of obtaining a relationship between a selected one of an applied force and an applied pressure, and a corresponding area of applanation of an eye. The method comprises the steps of applying to a surface of an eye a selected one of a variable force and a variable pressure to cause applanation of a portion of the surface of the eye; measuring a variable area of applanation of the surface of the eye corresponding to the selected one of a variable force and a variable pressure; and deducing at least one point of a relationship between the area of applanation of the eye and the selected one of a variable force and a variable pressure.

In one embodiment, the method further comprises the step of deducing an accurate value of intraocular pressure. In one embodiment, the method further comprises the step of displaying to a user of the apparatus an accurate value of intraocular pressure. In one embodiment, the relationship is deduced using a plurality of selected ones of a variable force and a variable pressure and a plurality of resulting areas of applanation. In one embodiment, the method further comprises the step of comparing the at least one point of a relationship of area of applanation of the eye corresponding to the selected one of an applied pressure and an applied force with at least one entry in the database comprising at least one relationship between a selected one of an applied pressure and an applied force and a corresponding area of applanation, each entry correlated with a condition of at least one eye. In one embodiment, the method further comprises the step of reporting an interpretive result indicative of a condition of the eye.

The foregoing and other objects, aspects, features, and advantages of the invention will become more apparent from the following description.

### BRIEF DESCRIPTION OF THE DRAWINGS

The objects and features of the invention can be better understood with reference to the drawings described below. The drawings are not necessarily to scale, emphasis instead generally being placed upon illustrating the principles of the invention. In the drawings, like numerals are used to indicate like parts throughout the various views.

FIG. 1 illustrates an exemplary embodiment of a system and a method for measuring the thickness of a cornea of an eye, according to the invention;

FIG. 2 is a diagram showing a simplified mechanical model intended to represent the elastic structures of the eyeball;

FIG. 3 is a diagram that shows in cross section, an idealized representation of an eyeball, including supporting ciliary muscles; and

FIG. 4 is a diagram showing three hypothetical curves, (a), (b), and (c), that illustrate relationships between applied force F and corresponding applanated area A, according to one aspect of the invention.

### DETAILED DESCRIPTION OF THE INVENTION

The invention provides systems and methods for obtaining more accurate and meaningful information about the condition of an eye, including providing a more reliable test for the presence of glaucoma than is presently available. In addition, the invention provides convenient systems and methods for monitoring the condition of a patient during treatment so that continuing effective care is provided.

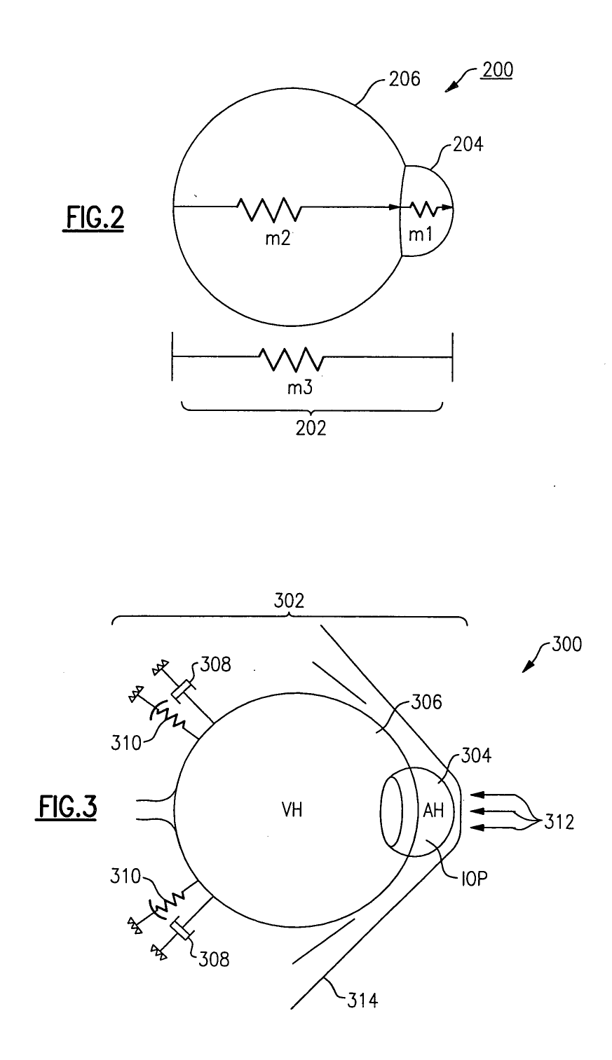

Turning to FIG. 2, there is shown a simplified mechanical model 200 intended to represent the elastic structures of an eye 202 having a cornea 204 and a substantially spherical scleral membrane 206. The anterior segment of the eye is represented as a linear spring of stiffness m1 and the posterior segment as a linear spring of stiffness m2, the two springs assumed to being connected in series. One may attempt to represent the eye 202 as the equivalent spring of stiffness m3, which in this model is the series combination of m1 and m2. However, this model may be over simplified since it does not account for the entire anatomy of the eyeball structures.

Turning to FIG. 3, there is shown in cross section an idealized diagram 300 depicting the anatomy of an eye 302. At the forward portion of the eye 302 is a cavity 304. Cavity 304 represents schematically the anterior chamber of the eye located between the cornea and the eye lens. The anterior chamber and the posterior chamber are filled with aqueous humor AH, which is a transparent fluid. The intraocular pressure (IOP_{I}) is a measure of the hydrostatic pressure exerted by the AH on the posterior corneal surface. It is this intraocular pressure that is significant from a clinical perspective.

At the rearward portion of the eye 302 is a cavity 306 that represents schematically the vitreous chamber of the human eye, and which is filled with a fluid called the vitreous humor VH, a fluid that is also transparent but that is more viscous than the aqueous humor AH. Also depicted in FIG. 3 are elements 308, 310 such springs and dashpots intended to represent structures, such as the supporting ciliary muscles, that apply forces to structures in the eye 302. The arrows 312 to the right of eye 302 are intended to represent a distributed force impinging on the eye 302. In addition, there are structures 314 around the eye, such as the socket in which eye 302 is located, that can apply forces to the eye 302.

It is in general assumed that the external pressure measured as IOP is that within the aqueous humor AH. However, as the above discussion of FIGS. 2 and 3 suggest, the response of the eye to an externally applied force is complex. Advantageously, the complete mechanical characterization of the eye need not be provided to improve estimation of IOP_{I}. If it is assumed that an externally applied force acts on the cornea 204 over an applanated area A, then IOP_{N} and IOP_{I} are expected to be related by:
IOP_{N}*A=IOP_{I}*A+R_{c} Eq. 1
where R_{c} indicates a force representing the resistance to deflection of the cornea. R_{c} is expected to vary with the thickness and viscoelastic properties of the cornea, and to first order is thought to scale linearly with the thickness of the cornea. The Goldmann apparatus and method do not account for variations in the value of R_{c} from individual to individual, thereby potentially resulting in inaccurate values of IOP_{I} for some individuals. Traditionally, this corneal thickness is measured at the center of the cornea, and is often discussed in the literature as the central corneal thickness (CCT). In light of the above description, the value of IOP that one wants to determine for purposes of diagnosis, monitoring, and treatment is given by:
IOP_{I}=IOP_{N}−R_{c}/A Eq. 2

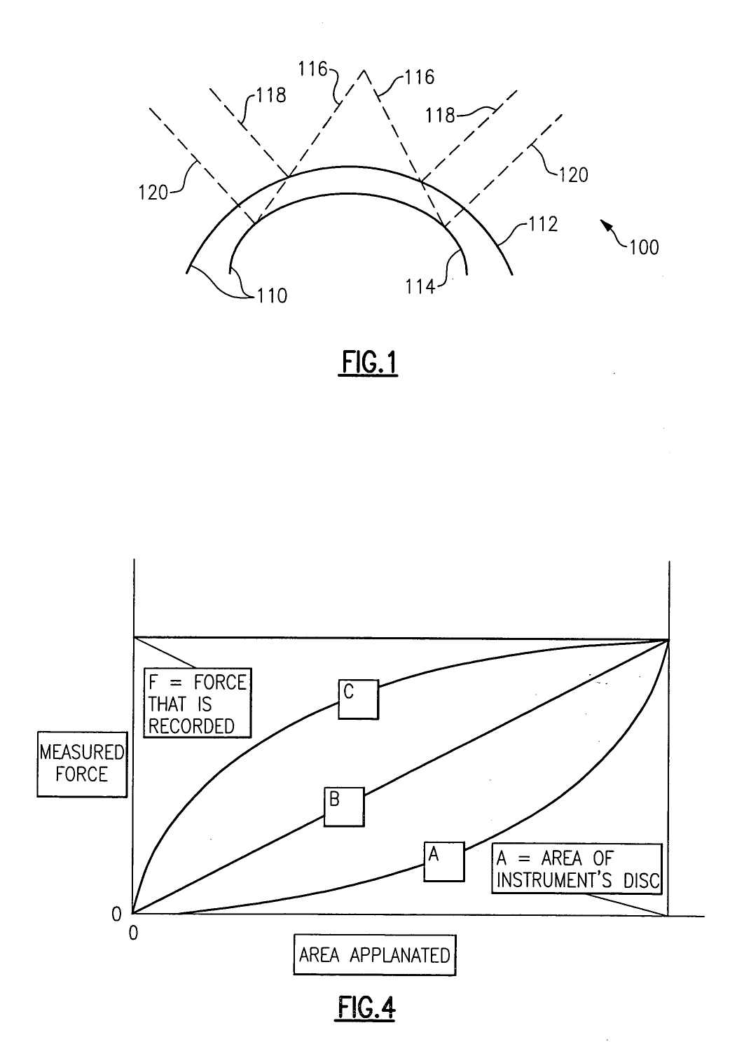

Since R_{c} depends on the thickness of the cornea, measurement of corneal thickness can be significant in estimating R_{c}. Turning to FIG. 1, there is depicted in diagram 100 an exemplary embodiment of a system and a method for measuring the thickness of a cornea 110 of an eye. The cornea 110 has an outer surface 112 and an inner surface 114. A plurality of preferably small diameter linearly polarized light beams 116, which in one embodiment are laser beams, are directed to the cornea 110. Beam 118 is reflected from the outer surface 112 and beam 120 is reflected from the inner surface 114. By monitoring the distance between the reflected light beams 118, 120, one can determine local variations in corneal thickness using geometrical optics. If the measurement is performed as a function of a vibration or a deflection induced in the cornea by application of force (such as by application of an air puff, by application of acoustic waves, or the like), one can also infer the thickness of the cornea by the linear or angular separation between the two reflected light beams using geometrical optics, and one can observe the change in displacement of the cornea as a function of the applied force. The applicants are aware of a paper entitled “Design and realization of a handheld-vibrometer system for non-contact in vivo detection of microvibrations of the human eye to determine the intraocular pressure (IOP),” by Gundlach, Rawer, Hey, Stork and Muller-Glasser, that appeared in Ophthalmic Technologies XII, proceedings of SPIE, Vol. 4611 (2002) at pages 9-19, a copy of which paper was appended to and made part of the disclosure of U.S. Provisional Patent Application Ser. No. 60/478,564 as Appendix A, the disclosure of which has been incorporated herein by reference in its entirety. This paper presents a description of measurement of an eye by causing a mechanical response in an eye by application of acoustical waves in the range of 100 to 1000 Hz, and optical sensing of the displacements induced in the surface of the eye by measurement with laser interferometry.

Furthermore, because of the interactions between the various elements making up the eye 302, one may expect that an excitation such as a pulse, that excites many natural frequencies in response to its application to the eye, will include a natural frequency f_{c} associated mostly with motion of the cornea relative to the remainder of the eye. Use of this frequency can provide insight to the value of the corneal thickness as well as to that of R_{c}.

As mentioned earlier, traditionally the Goldmann applanation method has been used to measure the force required to flatten a defined area of the cornea, commonly an area of 7.3542 mm². In the Goldmann method, it is the area to be applanated that is defined, and the force necessary to applanate the defined area is measured. With modern measurement and computation methods, neither the use of a fixed area selected to make computation easy for an operator (or to standardize a measurement), nor the use of a minimum force of the order of a gram to assure accuracy of measurement remain as limitations. Any area is suitable for computational purposes provided that it is measured with sufficient precision. Forces well below a gram and pressures below 1 mmHg are readily measured with precision using present day electronic apparatus such as strain gauges, mass flow controllers, and amplifiers. Force and pressure are related by F=P×A, where F is force, P is pressure, and A is area. It is possible to apply force, and thereby pressure, to an eye with a puff of air.

One method according to the principles of the invention of estimating IOP_{I} involves applying a defined or controlled force F or pressure P, and measuring either the force F or the pressure P and the applanation area A throughout an applanation test of an eye, as depicted in FIG. 4. In addition to the endpoint of the F versus A curve, the shape of the curve can offer information not previously available. In addition, the local slope and curvature of the measured curve are affected by corneal stiffness; these can be used to infer the contribution of R_{c} on the measured IOP_{N}. In FIG. 4, there are shown three hypothetical curves, (a), (b), and (c), of F (applied force) vs. A (applanated area). Curve (a) depicts a relationship wherein the applanation area A grows quickly for low force F application, and then more slowly as F increases. Conversely, curve (c) depicts a slow increase in A as F increases initially, and then more rapidly as F increases. Curve (b) shows a linear relationship between F and A. In FIG. 4, a curve represents a relationship between applied force F as the independent variable, with the applanated area A as the dependent variable, although this is counter to the normal presentation for independent and dependent variables. In some embodiments, one can take the area A as the independent variable, and the force F as the dependent variable. Similar graphs showing relationships between pressure P and area A can equally well be contemplated. The requirement that the area of applanation A be any specified value is eliminated; in fact, the area of applanation A is not the controlling variable as it is in the Goldmann method. While a single point (F/A) of the F versus A curves shown has historically been used as a measure of IOP in the Goldmann method, the curves themselves have not been, and are not, measured in standard medical practice. Up to now, measuring such curves has been impractical in a time short enough for a medical examination.

It is believed that observing, measuring, recording, and analyzing the relationship between applied force F (or applied pressure P) and resulting applanation area A, including observing the time evolution of the F−A (or P−A) relationship, can provide important information about the influence of the corneal thickness and stiffness on the value of IOP_{I}, as expressed by Eq. 2 above. This type of measurement is different from the Goldmann type measurement, wherein a fixed applanated area A=7.3542 mm² is used. In the measurement according to the invention, the area A expands from a smaller value (such as zero) to a final value (e.g., 7.3542 mm²). By measuring F (or alternatively, P), for example with strain gauges in the tonometer, and A, for example with a CCD camera, sampling and digitizing both signals, and recording the data so obtained, manipulation of the data with a programmed digital computer to extract information is straightforward. Present sensor, data conversion and computational technology allows such measurements to be made at rates of many samples per second. It is anticipated that combining information obtained from the shape of the F vs. A (or P vs. A) curve with data relating to selected point values (e.g., the force associated with an applanated area, which can differ from 7.3542 mm²) will allow the measured IOP_{N} to be corrected (i.e., to provide a measurement that more nearly represents IOP_{I}. In some embodiments, one can develop an applanation-force deflection curve to explore the behavior of the cornea and its stiffness. There is in principle no reason why meaningful measurements cannot be obtained for applanation areas in a range from greater than zero to at least the standard Goldmann area of 7.3542 mm², although areas greater than 7.3542 mm² can also provide useful information, provided that the force required to applanate an area, whether larger that 7.3542 mm² or not, is not excessive, and does not risk injury to an eye.

In one embodiment, a method for making such measurements includes the following steps. Data for the relationship between a selected one of an applied variable force F and an applied variable pressure P and applanation area A are studied for a sufficient sample of eyes. The data can include both static (or steady state) force-applanation data points, and dynamic data taken as a function of time, for example to detect, observe and record the time evolution or relaxation effects in the cornea, and to detect, observe and record viscoelastic effects attributable to the cornea. The data so obtained are correlated with the condition of each eye as measured by conventional methods. Based on the observed data, a database is constructed, comprising at least one entry of “normal” and “abnormal” F−A and/or P−A relationships, and “normal” and “abnormal” time-evolved responses of the cornea. In the course of measuring an eye for the purposes of medical diagnosis, or for monitoring the effects of a course of treatment or of the effects of time (e.g., aging) on an eye, the eye is subjected to a measurement of applanation area A as a function of applied variable force F or applied variable pressure P, with time evolution of the data as appropriate. The measured data for the eye are then compared to information in the database. Based at least in part on the comparison, and possibly including other information such as the age of the patient, and/or the medical history of the patient, an interpretive result is generated. As appropriate, and in compliance with regulations such as those in HIPPA, the interpretive result is provided to a practitioner.

The apparatus for making such a measurement includes a driver application section, and a measurement section. The driver application section applies a selected one of a variable pressure and a variable force to the surface of an eye. In some embodiments, the driver section uses direct mechanical pressure, or application of force or pressure using an air puff. In some embodiments, the driver application section is in communication with the measurement section, so that a limitation of the applied force or pressure, as measured by the measurement section, can be applied to the driver application section. In other embodiments, the force or pressure is limited exclusive of the measurement section, to avoid the possibility of injury to an eye. The measurement section includes a device to measure the applanated area, such as a CCD camera that provides a signal related to the applanated area, and can include a device to measure a response of a cornea, such as a corneal thickness measurement device, or a CCD camera that measures a shape of a region of a cornea and provides a signal representative of the observed shape. The apparatus includes a time clock, such as a crystal-controlled oscillator, for generating precise time intervals useful for measuring a time evolution of a signal. The apparatus further includes a programmable processor, such as a microprocessor; one or more computer program modules for controlling the operating of the device; and memory, such as machine readable storage media for recording the computer program modules, and for recording the results of operation of the apparatus. The apparatus can record data measured by the measurement section, including applied force F, applied pressure P, applanated area A, the time evolution of the F−A or P−A relationship, and the time behavior of an eye or of its parts, such as the cornea of an eye. The apparatus can be in communication with a machine-readable memory, such as a database, within which is recorded at least one entry comprising data relating to the F−A or P−A relationships for “normal” and “abnormal” conditions of an eye. The apparatus can be in communication with one or more input/output (“I/O”) devices, such as a keyboard, a pointing device such as a mouse, a touch screen, a touchpad, a microphone for receiving audible commands, a video display, a printer, and an audio signal enunciator such as a speaker. The I/O devices provide a user the capability to issue commands to the apparatus, and allow the apparatus to report the result of a measurement to a user. The I/O devices also allow the apparatus to report error conditions or requests for maintenance. The apparatus includes, as needed, analog-to-digital (A/D) and digital-to-analog (D/A) converters to convert a format of data from one form to another as required, as is common in the analog data acquisition and digital data processing arts.

In one embodiment, the rate of applanation or deflection varies, i.e., the speed of application of the force or the pressure is changed. It is believed that the cornea behaves in a viscoelastic manner. The force or pressure required to obtain a response of the cornea should then be a function of the rate of application of the force or pressure. By comparison, the resistance of a fluid, such as the aqueous humor, should not depend as significantly on the rate of application of a force or a pressure. Thus, the contributions of the two components, the aqueous humor and the cornea, can be separated by applying force at different rates and measuring the responses thereto.

Another embodiment to correct IOP_{N} for corneal contribution involves making multiple IOP_{N} measurements using contact or air puff methods at a selected number of different locations on the cornea, (for example, along a line that crosses the cornea in a selected direction, or at a series of points at known relative locations on two dimensions).

Another embodiment for estimating R_{c} involves measurement of a thickness of a cornea optically. In this embodiment, a high-speed camera is mounted laterally relative to the patient eye in combination with a standard applanation tonometer (such as the Perkins or air-puff). By synchronizing the tonometer and the camera, the camera records a sequence of frames using polarized monochromatic light as the cornea is deflected or applanated. Since corneal tissue is transparent, a dynamic cross-sectional view may be obtained. Once the corneal thickness is known, one can comparatively evaluate the deflection or applanation response (applied pressure versus deflection) with behavior predicted by either finite element analysis (FEA) or another suitable analytic model. Based on this comparison, one can infer IOP_{I} by subtraction as IOP_{I}=IOP_{N}−R_{c}/A.

Machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. New media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information.

Many functions of electrical and electronic apparatus can be implemented in hardware (for example, hard-wired logic), in software (for example, logic encoded in a program operating on a general purpose processor), and in firmware (for example, logic encoded in a non-volatile memory that is invoked for operation on a processor as required). The present invention contemplates the substitution of one implementation of hardware, firmware and software for another implementation of the equivalent functionality using a different one of hardware, firmware and software. To the extent that an implementation can be represented mathematically by a transfer function, that is, a specified response is generated at an output terminal for a specific excitation applied to an input terminal of a “black box” exhibiting the transfer function, any implementation of the transfer function, including any combination of hardware, firmware and software implementations of portions or segments of the transfer function, is contemplated herein.

While the present invention has been explained with reference to the structure disclosed herein, it is not confined to the details set forth and this invention is intended to cover any modifications and changes as may come within the scope and spirit of the following claims.

1. An apparatus for producing an accurate value of intraocular pressure of an eye, said apparatus comprising:
an intraocular pressure measuring device, said intraocular pressure measuring device providing a first intraocular pressure measurement of an eye;
a device for making a selected one of a corneal thickness measurement and at least one additional intraocular pressure measurement, separate and distinct from said first intraocular pressure measurement; and
a data analysis module that interrelates said first intraocular pressure measurement and said selected one of said corneal thickness measurement and said at least one additional intraocular pressure measurement;
whereby an accurate value of intraocular pressure is provided.

2. The apparatus of claim 1, further comprising a display module for displaying to a user of said apparatus said accurate value of intraocular pressure.

3. The apparatus of claim 1, wherein said data analysis module is further configured to generate an interpretive result indicative of a condition of said eye.

4. The apparatus of claim 3, further comprising a display module for displaying to a user of said apparatus said interpretive result indicative of a condition of said eye.

5. The apparatus of claim 1, wherein said data analysis module is further configured to generate an interpretive result useful to evaluate a condition of said eye.

6. The apparatus of claim 1, wherein said data analysis module is further configured to generate an interpretive result useful to manage a condition of said eye.

7. The apparatus of claim 1, wherein said intraocular pressure measuring device and said device for making said at least one additional intraocular pressure measurement are the same device.

8. The apparatus of claim 1, wherein said first intraocular pressure measurement and said at least one additional intraocular pressure measurement are intraocular pressure measurements performed on different regions of said eye.

9. The apparatus of claim 8, wherein said different regions comprise regions having different locations on a surface of said eye.

10. The apparatus of claim 8, wherein said different regions comprise regions having different surface areas on a surface of said eye.

11. The apparatus of claim 1, wherein said intraocular pressure measuring device is a device of the Goldmann type.

12. The apparatus of claim 1, wherein said intraocular pressure measuring device is adapted to measure intraocular pressure using a selected one of a first area and at least one additional area.

13. The apparatus of claim 1, wherein a change in applanated area with a change in a selected one of an externally applied force and an externally applied pressure is used for estimating intraocular pressure.

14. The apparatus of claim 1, wherein said intraocular pressure measuring device is a non-contact intraocular pressure measurement device.

15. The apparatus of claim 1, wherein said device for measuring said corneal thickness is a selected one of an apparatus passing light transversely through a cornea and an apparatus using a plurality of optical beams reflecting from different surfaces of a cornea.

16. A method of producing an accurate value of intraocular pressure of an eye, said method comprising the steps of:
measuring a first intraocular pressure of an eye;
measuring a selected one of a corneal thickness of said eye and at least one additional intraocular pressure of said eye, separate and distinct from said first intraocular pressure;
interrelating said first intraocular pressure and said selected one of said corneal thickness and said at least one additional intraocular pressure; and
providing an accurate value of intraocular pressure.

17. The method of claim 16, further comprising the step of displaying to a user of said method said accurate value of intraocular pressure.

18. The method of claim 16, further comprising the step of generating an interpretive result indicative of a condition of said eye.

19. The method of claim 18, further comprising the step of displaying to a user of said method said interpretive result indicative of a condition of said eye.

20. The method of claim 16, further comprising the step of generating an interpretive result useful to evaluate a condition of said eye.

21. The method of claim 16, further comprising the step of generating an interpretive result useful to manage a condition of said eye.

22. The method of claim 16, wherein said first intraocular pressure measurement and said at least one additional intraocular pressure measurement are performed on different regions of said eye.

23. The method of claim 22, wherein said different regions comprise regions having different locations on a surface of said eye.

24. The method of claim 22, wherein said different regions comprise regions having different surface areas on a surface of said eye.

25. The method of claim 16 wherein said intraocular pressure measurement is performed using a device of the Goldmann type.

26. The method of claim 16, wherein said intraocular pressure measurement is performed using a selected one of a first area and at least one additional area.

27. The method of claim 16, wherein a change in applanated area with a change in a selected one of an externally applied force and an externally applied pressure is used for estimating intraocular pressure.

28. The method of claim 16, wherein said intraocular pressure measurement is performed using a non-contact intraocular pressure measurement device.

29. The method of claim 16, wherein said measurement of said corneal thickness is performed using a selected one of a method in which light passes transversely through a cornea and a method using a plurality of optical beams reflecting from different surfaces of a cornea.

30. An apparatus for obtaining a relationship between a selected one of an applied force and an applied pressure, and a corresponding area of applanation of an eye, said apparatus comprising:
a driver application section for applying to a surface of an eye a selected one of a variable force and a variable pressure to cause applanation of a portion of said surface of said eye; and
a measurement section measuring said selected one of a variable force and a variable pressure, and measuring a corresponding variable area of applanation of said surface of said eye;
whereby said apparatus obtains at least one point of a relationship between said area of applanation of said eye and said selected one of a variable force and a variable pressure.

31. The apparatus of claim 30, further comprising an analysis module for deducing an accurate value of intraocular pressure.

32. The apparatus of claim 30, further comprising a display module for displaying to a user of said apparatus an accurate value of intraocular pressure.

33. The apparatus of claim 30, said apparatus further comprising a memory in communication with said measurement section, said memory configured to record a database comprising at least one entry, said entry representing a relationship between a selected one of an applied pressure and an applied force and a corresponding area of applanation, said entry correlated with a condition of at least one eye.

34. The apparatus of claim 33, said apparatus further comprising a processor for comparing said at least one point of a relationship of area of applanation of the eye corresponding to said selected one of an applied pressure and an applied force with at least one entry in said database comprising at least one relationship between a selected one of an applied pressure and an applied force and a corresponding area of applanation, each said entry correlated with a condition of at least one eye.

35. The apparatus of claim 34, said apparatus further comprising an output device for reporting an interpretive result indicative of a condition of said eye.

36. A method of obtaining a relationship between a selected one of an applied force and an applied pressure, and a corresponding area of applanation of an eye, said method comprising the steps of:
applying to a surface of an eye a selected one of a variable force and a variable pressure to cause applanation of a portion of said surface of said eye;
measuring a variable area of applanation of said surface of said eye corresponding to said selected one of a variable force and a variable pressure; and
deducing at least one point of a relationship between said area of applanation of said eye and said selected one of a variable force and a variable pressure.

37. The method of claim 36, further comprising the step of deducing an accurate value of intraocular pressure.

38. The method of claim 36, further comprising the step of displaying to a user of said apparatus an accurate value of intraocular pressure.

39. The method of claim 36, wherein said relationship is deduced using a plurality of selected ones of a variable force and a variable pressure and a plurality of resulting areas of applanation.

40. The method of claim 36, further comprising the step of comparing said at least one point of a relationship of area of applanation of the eye corresponding to said selected one of an applied pressure and an applied force with at least one entry in a database comprising at least one relationship between a selected one of an applied pressure and an applied force and a corresponding area of applanation, each said entry correlated with a condition of at least one eye.

41. The method of claim 40, further comprising the step of reporting an interpretive result indicative of a condition of said eye.
